(19) United States
(12) Patent Application Publication
(10) Pub. No.: US 2007/0232558 A1
(43) Pub. Date: Oct. 4, 2007

(54) METHODS AND COMPOSITIONS FOR TREATING PROSTATE CANCER USING DNA VACCINES DIRECTED TO CANCER TESTIS ANTIGEN

(76) Inventors: Douglas G. McNeel, Madison, WI (US); Jason Allan Dubovsky, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/693,856

(22) Filed: Mar. 30, 2007

Related U.S. Application Data
(60) Provisional application No. 60/787,808, filed on Mar. 31, 2006.

(51) Int. Cl. A61K 48/00; A61K 38/19

(52) U.S. Cl. 514/44; 424/85.1

(57) ABSTRACT

A DNA vaccine for the treatment of prostate cancer, comprising a plasmid vector comprising a nucleotide sequence encoding SSX-2 operably linked to a transcription regulatory element, wherein upon administration to a mammal a cytotoxic immune reaction against cells expressing SSX-2 is induced. In one embodiment, the SSX-2 encoded is a xenoantigen highly homologous to the autoantigen SSX-2 of the mammal. Also disclosed are methods for inducing immune reaction to SSX-2, or treating prostate cancer in a mammal, using the DNA vaccine and pharmaceutical compositions comprising the vaccine. In one embodiment, xenoantigen vaccination is followed by boosting with autoantigen SSX-2 from the same animal species as the mammal being treated.

### CROSS-REFERENCE TO RELATED APPLICATIONS

This application claims the benefit of U.S. Patent Application No. 60/787,808, filed on Mar. 31, 2006, which is herein incorporated by reference in its entirety.

### STATEMENT REGARDING FEDERALLY SPONSORED RESEARCH OR DEVELOPMENT

This invention was made with United States government support awarded by the following agency: NIH RR016489. The United States has certain rights in this invention.

### BACKGROUND OF THE INVENTION

Prostate cancer is a significant health risk for men over the age of 50, with 200,000 newly diagnosed cases each year (Jemal A. et al., Cancer Statistics, 2005 (2005) CA Cancer J Clin, 55:10-30). It is the most common tumor diagnosed among men and the second leading cause of male cancer-related death in the United States (Jemal et al., Cancer Statistics, 2003 (2003) CA Cancer J Clin, 53:5-26). Despite advances in screening and early detection, approximately 30% of patients undergoing definitive prostatectomy or ablative radiation therapy will have recurrent disease at 10 years (Oefelein et al., Long-term results of radical retropubic prostatectomy in men with high grade carcinoma of the prostate (1997) J Urol, 158:1460-1465). At present, there is no accepted adjuvant treatment for patients undergoing radical prostatectomy or ablative radiation therapy that has been shown to prevent the progression to metastatic disease. In addition to new treatments for metastatic disease, new strategies are needed to eradicate microscopic disease to prevent the progression to clinically apparent metastasis.

In patients who have undergone definitive ablative therapy for prostate cancer, the presence of detectable serum levels of prostate-specific antigen (PSA) has provided a valuable indicator of microscopic metastatic disease. In a retrospective review of 1,997 men treated with radical prostatectomy, 15% were found to have evidence of a PSA-only recurrence over a median 5-year follow up, so-called stage D0 disease (Pound et al., Natural history of progression after PSA elevation following radical prostatectomy (1999) JAMA 281:1591-7). Of these, 34% developed radiographically apparent metastatic disease, with a median time to development of metastatic disease of 8 years. In all patients with metastatic disease, the median time to death was 5 years (Pound et al., Natural history of progression after PSA elevation following radical prostatectomy (1999) JAMA 281:1591-7). These findings suggest that patients with stage D0 disease are at high risk for progressive disease, however with a long window of time to test adjuvant therapies. Similarly, many patients are found to have microscopic pelvic lymph node metastases at the time of radical prostatectomy, so-called stage D1 disease. At present, the best treatment for these patients is controversial, with some obtaining radical prostatectomy, others referred for radiation therapy with or without androgen deprivation therapy, and yet others are expectantly observed without specific treatment. In retrospective studies, 10-year disease-specific recurrence and mortality is on the order of 50 to 66% for patients with stage D1 disease (Sgrignoli et al., Prognostic factors in men with stage D1 prostate cancer: identification of patients less likely to have prolonged survival after radical prostatectomy (1994) J Urol, 152:1077-81; and Cadeddu et al., Stage D1 (T1-3, N1-3, M0) prostate cancer: a case-controlled comparison of conservative treatment versus radical prostatectomy (1997) Urology, 50:251-5). This high-risk stage of minimal residual disease also provides an opportunity to test novel adjuvant therapies.

Immunological therapies, and vaccines in particular, are appealing as possible treatment options for prostate cancer for several reasons. Such therapies may be relatively safe and inexpensive treatments compared with chemotherapies for a disease for which no standard adjuvant treatments exist (Kent et al., Immunity of prostate specific antigens in the clinical expression of prostatic carcinoma (1976) In: Crispen R G, ed. Neoplasm immunity: mechanisms. Chicago, ITR, pp. 85-95; Guinan et al., Immunotherapy of prostate cancer: a review (1984) Prostate, 5:221-230; and McNeel et al., Tumor vaccines for the management of prostate cancer (2000) Arch. Immunol. Ther. Exp., 48:85-93). Moreover, prostate cancer is a slow-growing disease, with typically over five years from the time of diagnosis of organ-confined disease to the development of clinically apparent metastatic disease. Such a slow-growing disease might be more amenable to vaccine-based treatments than a rapidly growing tumor, assuming that microscopic amounts of disease would be easier to treat than bulky or rapidly growing disease by vaccines. In fact, vaccines have already entered clinical trials for prostate cancer targeting a variety of prostate-specific proteins, with at least two dendritic cell-based vaccines suggesting clinical benefit in patients with low-volume metastatic disease (Murphy et al., Phase II prostate cancer vaccine trial: report of a study involving 37 patients with disease recurrence following primary treatment (1999) Prostate, 39:54-59; and Small et al., Immunotherapy of hormone-refractory prostate cancer with antigen-loaded dendritic cells (2000) J. Clin. Oncol., 18:3894-3903).

The use of plasmid DNA alone as a means of in vivo gene delivery by direct injection into muscle tissue was first described by Wolff et al. (Wolff et al., Direct gene transfer into mouse muscle in vivo (1990) Science, 247:1465-1468). It was subsequently found that intramuscular or intradermal administration of plasmids expressing foreign genes elicited immune responses (Tang, et al., Genetic immunization is a simple method for eliciting an immune response (1992) Nature, 356:152-154; Wang et al., Gene inoculation generates immune responses against human immunodeficiency virus type 1 (1993) Proc Natl. Acad. Sci. USA, 90:4156-4160; and Raz et al., Intradermal gene immunization: the possible role of DNA uptake in the induction of cellular immunity to viruses (1994) Proc Natl. Acad. Sci. USA, 91:9519-9523). This has quickly led to numerous investigations into the use of plasmid DNA as a means of vaccine antigen delivery, both in animal and human models. DNA vaccines, like peptide-based vaccines, are advantageous in being relatively easy and inexpensive to manufacture, and are not individualized for patients as are dendritic cell-based vaccines. Unlike recombinant protein vaccines, in which the antigen is taken up by antigen presenting cells and expressed predominantly in the context of MHC class II, animal studies have demonstrated that DNA in nucleic acid vaccines is taken up and expressed by antigen-presenting cells directly, leading to antigen presentation through naturally processed MHC class I and II epitopes (Iwasaki, et al. The dominant role of bone marrow-derived cells in CTL induction following plasmid DNA immunization at different sites (1997) J Immunol, 159:11-14). This method of immunization is consequently similar to the use of viral immunization vectors, however without the additional foreign antigens introduced with a viral vector and therefore with less risk of an overwhelming immune response to the vector itself (Irvine et al. The next wave of recombinant and synthetic anticancer vaccines (1995) Seminars in Canc. Biol. 6:337-347). This direct expression by host cells, including MHC class I expressing bystander cells, has been demonstrated to lead to vigorous CD8+CTL responses specific for the targeted antigen (Iwasaki et al., The dominant role of bone marrow-derived cells in CTL induction following plasmid DNA immunization at different sites (1997) J. Immunol. 159:11-14; Chen et al., Induction of CD8+T cell responses to dominant and subdominant epitopes and protective immunity to Sendai virus infection by DNA vaccination (1998) J. Immunol., 160:2425-2432; Thomson et al., Delivery of multiple CD8 cytotoxic T cell epitopes by DNA vaccination (1998) J. Immunol., 160:1717-1723; and Cho et al., Immunostimulatory DNA-based vaccines induce cytotoxic lymphocyte activity by a T-helper cell-independent mechanism (2000) Nat. Biotechnol. 18:509-514). In addition, plasmid DNA used for immunization may potentially stay present within cells at the site of immunization, providing a constant source of antigenic stimulation, rather than protein or peptide vaccines that are rapidly cleared by the reticuloendothelial system (Wolff et al., Direct gene transfer into mouse muscle in vivo. (1990) Science, 247:1465-1468; and Tighe et al., Gene vaccination: plasmid DNA is more than just a blueprint (1998) Immunol. Today, 19:89-97). It has been suggested that persistent antigen expression may lead to long-lived immunity (Raz et al., Intradermal gene immunization: the possible role of DNA uptake in the induction of cellular immunity to viruses (1994) Proc. Natl. Acad. Sci. USA, 91:9519-9523).

Recently, clinical trials have suggested that plasmid DNA vaccines are safe and immunologically effective in humans. Boyer and colleagues reported that doses of 300 μg of plasmid DNA encoding HIV rev and env proteins administered intramuscularly were capable of eliciting antigen-specific, IFNγ-secreting T cell responses in HIV-seronegative patients (Boyer et al., Vaccination of seronegative volunteers with a human immunodeficiency virus type 1 env/rev DNA vaccine induces antigen-specific proliferation and lymphocyte production of β-chemokines (2000) J. Infect. Dis. 181:476-83). In addition, results of a clinical trial targeting prostate-specific membrane antigen (PSMA) in patients with prostate cancer by means of plasmid DNA and adenovirus have been reported (Mincheff et al., Naked DNA and Adenoviral Immunizations for Immunotherapy of Prostate Cancer: A Phase VI/II Clinical Trial (2000) Eur. Urol., 38:208-217). In this study, 26 patients were immunized either in a prime/boost strategy with an adenoviral vector expressing PSMA followed by immunization with plasmid DNA expressing PSMA, or with plasmid DNA alone. The authors report no significant toxicity with doses of 100-800 μg of plasmid DNA administered intradermally, and suggest that patients receiving plasmid DNA expressing PSMA and CD86 with soluble GM-CSF as an adjuvant were all successfully immunized (Mincheff et al., Naked DNA and Adenoviral Immunizations for Immunotherapy of Prostate Cancer: A Phase I/II Clinical Trial (2000) Eur. Urol., 38:208-217).

A DNA vaccine for the treatment of prostate cancer based on prostatic acid phosphatase (PAP), which is expressed almost exclusively in normal and malignant prostate tissues, has also been described (US 2004/0142890).

Cancer testis antigens (CTAs) such as synovial sarcoma X breakpoint 2 (SSX-2) are a class of proteins which are exclusively expressed in testis tissues and often aberrantly in multiple forms of cancers and thus they have the potential to serve as targets for cancer treatment.

### BRIEF SUMMARY OF THE INVENTION

The invention provides a method for inducing an immune reaction to SSX-2 in a mammal in need thereof, the method comprising administering to the mammal an effective amount of a recombinant DNA construct comprising a polynucleotide sequence encoding an SSX-2 polypeptide or an immunogenic fragment thereof operatively linked to a transcriptional regulatory element, whereby the mammal develops an immune reaction against SSX-2. In a preferred embodiment, the mammal, preferably a human, is a prostate cancer patient.

Preferably, the polynucleotide sequence encoding an SSX-2 polypeptide is a human SSX-2 gene. In another embodiment, the polynucleotide sequence encoding SSX-2 is a rat or mouse SSX-2 gene.

According to the invention, the recombinant DNA construct is administered to the mammal intradermally, intramuscularly or intravascularly, including intravenously and intraarterially.

The method according to the present invention induces a cytotoxic immune reaction against cells expressing SSX-2. Preferably, both humoral and cellular immune reactions against SSX-2 are induced.

In one embodiment, the method of the present invention employs a “prime-boost” strategy, which comprises administering to the mammal an effective amount of a first recombinant DNA construct comprising a first polynucleotide sequence encoding a first SSX-2 polypeptide or an immunogenic fragment thereof operatively linked to a transcriptional regulatory element; followed by administering to the mammal an effective amount of a second recombinant DNA construct comprising a second polynucleotide sequence encoding a second SSX-2 polypeptide or an immunogenic fragment thereof operatively linked to a transcriptional regulatory element; wherein the first polynucleotide sequence and the second polynucleotide molecule originate from two different animal species, whereby an immune reaction against SSX-2 is induced in the mammal. In one form, the first polynucleotide sequence originates from an animal species other than the mammal, and the second polynucleotide sequence originates from the same animal species as the mammal. Preferably, the mammal is a human, and the first polynucleotide sequence encoding SSX-2 originates from a rat or mouse. In another form, the second polynucleotide sequence originates from the same animal species as the mammal, and the first polynucleotide sequence encodes an SSX-2 polypeptide that shares at least 85%, preferably at least 88%, still more preferably at least 90%, more preferably at least 95%, and most preferably at least 98% homology to the first SSX-2 polypeptide.

According to another aspect of the present invention, a DNA vaccine is contemplated which comprises a plasmid vector comprising a polynucleotide sequence encoding an SSX-2 polypeptide or an immunogenic fragment thereof operably linked to a transcription regulatory element, wherein upon administration to a mammal a cytotoxic immune reaction against cells expressing SSX-2 is induced in the mammal. The vaccine of the present invention preferably is suitable for intradermal, intramuscular or intravascular administration to a human. According to a preferred embodiment, the plasmid vector comprises a backbone of pNGVL3, a polynucleotide sequence encoding an SSX-2 polypeptide or an immunogenic fragment thereof operably inserted therein, and one or a plurality of an immuno-stimulatory sequence (ISS) motif.

Preferably, the DNA vaccine according to the invention comprises a plasmid vector that comprises a polynucleotide sequence encoding an SSX-2 polypeptide or an immunogenic fragment thereof operatively linked to a CMV promoter; a CMV intron A operatively linked to the polynucleotide sequence encoding the SSX-2 polypeptide or an immunogenic fragment thereof for enhancing expression of the polynucleotide sequence; and at least one copy of an immuno-stimulatory fragment comprising 5′-GTCGTT-3′. In one embodiment, the plasmid construct does not express in eukaryotic cells any gene other than the polynucleotide sequence encoding the SSX-2 polypeptide or an immunogenic fragment thereof. The plasmid vector pTVG4 is particularly preferred.

Also disclosed are pharmaceutical compositions comprising the DNA vaccine of the invention, and a pharmaceutically acceptable carrier. Preferably, the pharmaceutical composition of the invention comprises the DNA vaccine and further a suitable amount of immuno-stimulant such as GM-CSF, optionally with a pharmaceutically acceptable carrier.

A kit containing the DNA vaccine of the invention and an instruction manual directing administering the vaccine to a mammal having prostate cancer (e.g., a human prostate cancer patient) is also within the scope of the invention.

### BRIEF DESCRIPTION OF THE SEVERAL VIEWS OF THE DRAWINGS

FIG. 1 shows that sequence confirmed phage encoding cancer-testis antigens showed mRNA expression by RTPCR.

FIG. 2 shows that high throughput immunoblot (HTI) methodology provides an efficient means of quantitatively mass screening sera for reactivity to multiple antigens.

FIG. 3 shows that HTI identified significant IgG responses to at least 2 of 29 known CTAs in sera from prostate cancer patients.

FIG. 4A shows that SSX-2 displays testis-specific expression among normal tissues.

FIG. 4B and FIG. 4C show that SSX-2 is expressed at human prostate cancer metastatic sites and prostate cancer cell lines.

FIG. 5 depicts the plasmid map of pTVG4.

FIG. 6 shows that cytotoxic T cells specific for an HLA-A2 peptide epitope presented by SSX-2 can be cultured from the peripheral blood of a prostate cancer patient.

### DETAILED DESCRIPTION OF THE INVENTION

This invention provides pharmaceutical compositions and methods using plasmid DNA vaccines for the treatment of prostate cancer. Specifically, this invention provides recombinant plasmid vectors comprising genes or polynucleotide molecules encoding one or more prostate cancer CTAs (CTAs that are expressed in prostate cancer cells) such as SSX-2 or an immunogenic fragment thereof for preventing or treating prostate cancer, including metastatic tumors thereof.

The term SSX-2 is used broadly in the specification and claims to refer to the human SSX-2 or a homologue thereof from another species such as another mammalian species. For example, the term “rat SSX-2” is used in the specification and claims to refer to the human SSX-2 homologue in rat as provided under GenBank Accession Number BC100112. Similarly, the term “mouse SSX-2” is used in the specification and claims to refer to the human SSX-2 homologue in mouse as provided under GenBank Accession Number NM_{—}001001450.

The vaccines of the present invention, when directly introduced into a vertebrate in vivo, including mammals such as humans, induce the expression of encoded polypeptides within the animal, and cause the animal's immune system to become reactive against the polypeptides. The vaccines may be any polynucleotides that are capable of generating immune responses to an encoded polypeptide. The vaccines are referred to herein as polynucleotide vaccines. Preferably, the polynucleotide vaccines of the present invention are DNA vaccines, especially plasmid DNA vaccines.

The instant invention also provides a method for using a polynucleotide which, upon introduction into a vertebrate, induces the expression, in vivo, of the polynucleotide thereby producing the encoded polypeptide, and causes the vertebrate to become immune reactive against the polypeptide so produced.

DNA vaccines, like peptide-based vaccines, are advantageous in being relatively easy and inexpensive to manufacture, and are not individualized for patients, as are dendritic cell-based vaccines. Unlike recombinant protein vaccines, in which the antigen is taken up by antigen presenting cells and expressed predominantly in the context of MHC class II, DNA in nucleic acid vaccines is taken up and expressed by antigen-presenting cells directly, leading to antigen presentation through both naturally processed MHC class I and II epitopes (Iwasak et al., 1997, J. Immunol. 159:11-4).

Given their potential ability to elicit antigen-specific cytotoxic T-cell (CTL) immunity in an MHC class I diverse population, DNA-based vaccines for various diseases have recently entered human clinical trials (Mincheffet al., 2000, Eur. Urol., 38:208-217). This method of immunization is similar to the use of viral immunization vectors, but without the additional foreign antigens introduced with a viral vector and therefore with less risk of an overwhelming immune response to the vector itself (Irvine and Restifo, 1995, Seminars in Canc. Biol. 6:337-347). Direct expression by host cells, including MHC class I-expressing bystander cells, has been demonstrated to lead to vigorous CD8+CTL responses specific for the targeted antigen (Iwasak et al., 1997, J. Immunol. 159:11-4; Chen et al., 1998, J. Immunol. 160:2425-2432; Thomson et al., 1998, J. Immunol. 160:1717-1723; and Cho et al., 2000, Nat. Biotechnol, 18:509-14). In addition, plasmid DNA used for immunization may remain within cells at the site of immunization, providing a constant source of antigenic stimulation, rather than protein or peptide vaccines that are rapidly cleared by the reticuloendothelial system (Wolff et al., 1990, Science 247:1465-8; and Tighe et al., 1998, Immunol. Today 19:89-97). Persistent antigen expression may lead to long-lived immunity (Raz et al., 1994, Proc. Natl. Acad. Sci. USA 91:9519-23).

The present invention provides DNA-based vaccines that express a protein antigen, SSX-2, or an immunogenic fragment thereof, and methods for treating prostate cancers in an animal using the vaccines. In addition to the reasons explained above, plasmid vaccines are advantageous over viral vaccines. For example, viral vaccines are not amenable to repeated immunizations. With viral vectors, one is trying to elicit an immune response against a “self” protein encoded by a foreign virus. The immune system preferentially recognizes the foreign proteins, sometimes hundreds of proteins, encoded by the virus. For example, the present inventor has found in rats that repeated immunizations with a vaccinia virus encoding human PAP (hPAP) elicits a strong vaccinia response but no hPAP-specific response. That same finding has now been shown in humans, in a trial in which repeated immunization with the vaccinia virus encoding human PSA elicited weak PSA-specific immunity, but potent vaccinia immunity (Sanda et al., 1999, Urology 53:260). The direction in the field of viral-based vaccines is to “prime” with a virus encoding the antigen, and then “boost” with a different virus (like adenovirus or fowl pox) encoding the same antigen. The advantage of plasmid DNA vaccines is that they encode a defined, often small, number of proteins. Therefore, one can repetitively immunize the animal or patient. Furthermore, a virus may kill cells, incorporate into the genome, or potentially induce other unwanted immune responses. All these are disadvantages that are likely avoided by DNA plasmid vaccines.

It is readily recognizable that an SSX-2 gene of any origin, or any of its derivatives, equivalents, variants, mutants etc., is suitable for the instant invention, as long as the polypeptide or protein encoded by the genes, or derivatives, equivalents, variants, or mutants thereof are able to induce an immune reaction in the host animal substantially similar to that induced by an autoantigenic or xenoantigenic SSX-2 protein in the animal.

The human SSX-2 gene and its homologues are known. The human SSX-2 cDNA sequence (SEQ ID NO:1) and amino acid sequence (SEQ ID NO:2) can be found at GenBank Accession Number BC007343. The cDNA sequence (SEQ ID NO:3) and the amino acid sequence (SEQ ID NO:4) of the SSX-2 homologue from rat can be found at GenBank Accession Number BC100112. The cDNA sequence (SEQ ID NO:5) and the amino acid sequence (SEQ ID NO:6) of the SSX-2 homologue from mouse can be found at GenBank Accession Number NM_{—}001001450. As will be readily recognized by one of ordinary skill in the art, any DNA sequence that encode one of the above three amino acid sequences are suitable for the present invention, and any other SSX-2 homologues from other animals, as they become identified, characterized, and cloned are also suitable for the present invention.

As is well-known to those skilled in the art, polypeptides having substantial sequence similarities cause identical or very similar immune reaction in a host animal. As discussed below, this phenomenon is the basis for using a xenoantigen for inducing autoreactive reaction to an otherwise tolerated autoantigen. Accordingly, any DNA sequences encoding a derivative, equivalent, variant, fragment, or mutant of any of the known or to-be-identified SSX-2 polypeptides or proteins is also suitable for the present invention. The polypeptides encoded by these DNA sequences are useful as long as the polypeptides encoded by these are structurally similar to the autologous SSX-2, and are sufficiently immunogenic.

It is readily apparent to those ordinarily skilled in the art that variations or derivatives of the nucleotide sequence encoding the polypeptide or protein antigen can be produced which alter the amino acid sequence of the encoded protein. The altered expressed polypeptide or protein may have an altered amino acid sequence, for example by conservative substitution, yet still elicits immune responses which react with the protein antigen, and are considered functional equivalents. According to a preferred embodiment, the derivative, equivalents, variants, fragments or mutants of an SSX-2 are polypeptides that are at least 85% homologous to the human SSX-2 sequence of SEQ ID NO:2. More preferably, the homology is at least 88%, preferably at least 90%, still more preferably at least 95%, and still more preferably at least 98%.

As used in this application, “percent identity” between amino acid or nucleotide sequences is synonymous with “percent homology,” which can be determined using the algorithm of Karlin and Altschul (Proc. Natl. Acad. Sci. USA 87, 2264-2268, 1990), modified by Karlin and Altschul (Proc. Natl. Acad. Sci. USA 90, 5873-5877, 1993). The noted algorithm is incorporated into the NBLAST and XBLAST programs of Altschul et al. (J. Mol. Biol. 215, 403-410, 1990). BLAST nucleotide searches are performed with the NBLAST program, score=100, wordlength=12, to obtain nucleotide sequences homologous to a polynucleotide of the invention. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (Nucleic Acids Res. 25, 3389-3402, 1997). When utilizing BLAST and Gapped BLAST programs, the default parameters of the respective programs (e.g., XBLAST and NBLAST) are used.

As used herein, the term “conservative substitution” denotes the replacement of an amino acid residue by another, biologically similar residue. It is well known in the art that the amino acids within the same conservative group can typically substitute for one another without substantially affecting the function of a protein. For the purpose of the present invention, such conservative groups are set forth in Table 1 based on shared properties.

TABLE 1
Conservative substitution.

| | |
|---|---|
| Original Residue | Conservative Substitution |

| | |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, Phe, Ala |

In addition, fragments of the full-length genes which encode portions of the full-length protein may also be constructed. These fragments may encode a protein or polypeptide which elicits humoral immune responses, cellular (including cytotoxic) immune responses, or both, against the protein antigen, and are considered functional equivalents.

The SSX-2 gene is preferably ligated into an expression vector which has been specifically optimized for polynucleotide vaccinations. Elements include a transcriptional promoter, immunogenic epitopes, and additional cistrons encoding immunoenhancing or immunomodulatory genes, with their own promoters, transcriptional terminator, bacterial origin of replication and antibiotic resistance gene, as well known to those skilled in the art. Optionally, the vector may contain internal ribosome entry sites (IRES) for the expression of polycistronic mRNA.

In one embodiment of this invention, a gene encoding an SSX-2 polypeptide or protein is directly linked to a transcriptional promoter. The use of tissue-specific promoters or enhancers, for example the muscle creatine kinase (MCK) enhancer element may be desirable to limit expression of the polynucleotide to a particular tissue type. For example, myocytes are terminally differentiated cells which do not divide. Integration of foreign DNA into chromosomes appears to require both cell division and protein synthesis. Thus, limiting protein expression to non-dividing cells such as myocytes may be preferable. In addition, a PSA promoter may be used to limit expression of the protein to prostate tissue. In one embodiment, tissue- or cell-specific promoters may be used to target the expression of the protein to antigen-presenting cells. For example, an a-fetoprotein (AFP) promoter (see e.g., Peyton et al. 2000, Proc. Natl. Acad. Sci., USA. 97:10890-10894) may be used to limit expression to liver tissues. However, use of the CMV promoter is adequate for achieving expression in many tissues into which the plasmid DNA vaccine is introduced.

Suitable vectors include any plasmid DNA construct encoding an SSX-2 antigen or a functional equivalent or derivative thereof, operatively linked to a eukaryotic promoter. Examples of such vectors include the pCMV series of expression vectors, commercially available from Stratagene (La Jolla, Calif.); or the pCDNA or pREP series of expression vectors by Invitrogen Corporation (Carlsbad, Calif.).

A preferred vector is pNGVL3 available from the National Gene Vector Laboratory at the University of Michigan. This vector, similar to the pCDNA3.1 eukaryotic expression vector of Invitrogen Corp. (Carlsbad, Calif.), drives transcription from the CMV promoter, but also includes the CMV intron A sequence to enhance protein expression (Lee et al., 1997, Mol. Cells 7:495-501). The vector also contains a multi-cloning site, and does not express a eukaryotic antibiotic resistance gene, such that the only protein expression expected in a eukaryotic system is the one driven from the CMV promoter, unlike the pCDNA vector. Another preferred vector is the pTVG4 vector described in US 2004/0142890, which is herein incorporated by reference in its entirety. The pTVG4 vector can be made by incorporating 2 copies of a 36-bp immunostimulatory (ISS) fragment containing the 5′-GTCGTT-3′ motif previously identified (Hartmann et al., 2000, J. Immunol. 164:1617-24) into pNGVL3.

There are many embodiments of the instant invention which those skilled in the art can appreciate from the specification. Thus, different transcriptional promoters, terminators, and other transcriptional regulatory elements may be used successfully. Examples of other eukaryotic transcription promoters include the Rous sarcoma virus (RSV) promoter, the simian virus 40 (SV40) promoter, the human elongation factor-1α (EF-1α) promoter, and the human ubiquitin C (UbC) promoter.

The vectors of the present invention may be delivered intradermally, intramuscularly, or intravascularly (including intraarterially). In preferred embodiments, delivery may be a combination of two or more of the various delivery methods.

In a particularly preferred embodiment, the animal in need of treatment may be first primed with a vector of the present invention comprising a xenoantigen, followed by a boosting with another vector comprising the same or a different xenoantigen, or an autoantigen, to achieve a robust and long lasting immune response against prostate cells. Both priming and boosting may be by any of the intradermal, intramuscular and intravascular delivery methods.

“Naked” plasmid DNA expressing a transgene could be directly injected intradermally or intramuscularly, taken up, and expressed (see e.g., Wolff et al., 1990, Science 247:1465-8). The efficiency of this approach may be low, with only a small percentage of myocytes being directly transformed in vivo, and within only a limited area of muscle tissue targeted by this directed delivery. Various alternative approaches yielding a higher gene delivery efficiency are known (see e.g., Acsadi et al., 1991, New Biol. 3:71-81). Subsequent work on strategies that increase uptake of plasmid DNA by muscle tissue focused on various carrier solutions and molecules (Wolff et. al., 1991, Biotechniques 11:474-85; and Budker et. al., 1996, Nat. Biotechnol. 14:760-4), the use of myotoxic agents to enhance DNA uptake (Davis et al., 1993, Hum. Gene Ther. 4:151-9; and Danko et al., 1994, Gene Ther. 1:114-21), and the use of various transcriptional promoters and plasmid DNA backbones (Manthorpe et al., 1993, Hum. Gene Ther. 4:419-31).

In a preferred embodiment, plasmid vectors of the present invention may be delivered to the patient in need thereof intravascularly. Plasmid DNA delivered intravascularly resulted in 100-fold higher uptake in downstream tissues in rodent studies (Budker et al., 1996, Gene Ther. 3:593-8). Intravascular delivery may be intravenal, e.g. by direct injection of plasmid DNA into the portal vein of rodents with uptake and expression demonstrated in hepatocytes (Budker et al., 1996, Gene Ther. 3:593-8; and Zhang et al., 1997, Hum. Gene Ther. 8:1763-72). Intravascular delivery may also be performed more directly by intraarterial delivery. For example, initial studies in rodents demonstrated that high levels of gene expression in hind limb muscle could be obtained by rapid injection of plasmid DNA into the femoral artery (Budker et al., 1998, Gene Ther. 5:272-276). This approach is efficient and safe in non-human primates as well, with an average of 7% of downstream myofibers expressing a β-galactosidase reporter construct two weeks after intraarterial DNA administration (Zhang et al., 2001, Hum. Gene Ther. 12:427-438). Parallel studies in T cell immuno-suppressed rats showed that gene expression was stable for at least 10 weeks (Zhang et al., 2001, Hum. Gene Ther. 12:427-438).

Accordingly, delivery of plasmid DNA vaccines of the present invention can be done by direct intraarterial administration. This method provides effective delivery to MHC class I expressing cells. Administrations of plasmid DNA vaccines intravascularly can result in increased antigen expression and subsequently lead to enhanced immune responses, and increased antigen expression in MHC class I expressing cells by means of intraarterial delivery of DNA plasmid may lead to a more robust immune response with SSX-2-specific CTL. An intraarterial method of DNA delivery may be at least as effective or more effective than traditional intradermal administration of DNA in eliciting SSX-2-specific immunity.

In another embodiment, intravenous delivery may also be used, employing methods well known to those skilled in the art (See e.g., Budker et al., 1998, Gene Ther. 5:272-276; and Budker et al., 1996, Gene Ther. 3:593-598). This delivery method may lead to a high level of antigen expression in hepatocytes. Expression of the antigen in liver, a tissue more rich with antigen-presenting cells, may lead to a more pronounced Th1/CTL response than expression in muscle tissue.

The DNA vaccines of the present invention are preferably used in a prime-boost strategy to induce robust and long-lasting immune response to SSX-2. Priming and boosting vaccination protocols based on repeated injections of the same antigenic construct are well known and result in strong CTL responses. In general, the first dose may not produce protective immunity, but only “primes” the immune system. A protective immune response develops after the second or third dose.

In one embodiment, the DNA vaccines of the present invention may be used in a conventional prime-boost strategy, in which the same antigen is administered to the animal in multiple doses. In a preferred embodiment, the DNA vaccine is used in one or more inoculations. These boosts are performed according to conventional techniques, and can be further optimized empirically in terms of schedule of administration, route of administration, choice of adjuvant, dose, and potential sequence when administered with another vaccine, therapy or homologous vaccine (such as a plasmid DNA encoding a rat or mouse SSX-2 gene).

The DNA vaccines of the present invention are preferably used in a prime-boost strategy using an alternative administration of xenoantigen- and autoantigen-encoding vectors. Specifically, according to the present invention, the animal is first treated, or “primed,” with a DNA vaccine encoding an antigen of foreign origin (a “xenoantigen”). Subsequently, the animal is then treated with another DNA vaccine encoding an antigen which is corresponding to the xenoantigen, but is of self origin (“autoantigen”). This way, the immune reaction to the antigen is boosted. The boosting step may be repeated one or more times.

A xenoantigen, as compared to a self-antigen or an autoantigen, is an antigen originated in or derived from a species different from the species that generates an immune reaction against the antigen. Xenoantigens usually are highly homologous molecules to a corresponding autoantigen. Xenoantigens have been shown to be able to elicit auto-reactive immunity. For example, molecular mimicry by highly homologous viral antigens has been one theory to explain the occurrence of several autoimmune diseases (von Herrat and Oldstone, 1996, Curr. Opin. Immunol. 8:878-885; and Oldstone, 1998, Faseb J. 12:1255-1265). That is, the induction of immune responses following infection by viral antigens that closely resemble normal autologous proteins may then lead to an autoimmune reaction to the autologous protein.

The use of highly homologous foreign antigens or xenoantigens as vaccine antigens to elicit autoreactive immunity has been explored in animal models. For example, xenoantigens derived from zona pellucida of foreign species can elicit autoreactive T cell responses and disrupt ovarian function in a variety of animal species studied (Mahi-Brown et al., 1992, J. Reprod. Immunol. 21:29-46; and Mahi-Brown, 1996, J. Reprod. Fertil. Suppl. 50:165-74). While not wishing to be bound by any theory on mechanism, it is believed that because T cells involved in autoimmune processes recognize peptide epitopes presented in the context of MHC molecules, the uptake and MHC presentation of a homologous foreign antigen presumably exposes T cell epitopes with enhanced MHC binding or unmasks cryptic epitopes of the native antigen not normally recognized.

While the prime-boost strategy is known to work with antigens of different origins, it is readily apparent to those ordinarily skilled in the art that variants, derivatives or equivalents, as discussed above, of the nucleotide sequence encoding a self-antigen can also be used to achieve the same results as xenoantigens.

The invention will be more fully understood upon consideration of the following non-limiting examples.

### EXAMPLE 1

### Identification of Cancer-Testis Antigen SSX-2 as a Prostate Cancer Antigen

Methods

High Throughput Immunoblot (HTI): 10,000 pfu pTripleX phage were mechanically spotted in a 9×8 grid pattern onto XL-1 blue Top Agarose lawns in Nunc OmniTray LB agarose plates using a Biomek FX liquid handling robot. After a short drying period precut IPTG infused nitrocellulose filters were layered on each plate and incubated at 37° C. to allow phage proliferation. The filters were washed and then blocked and incubated with human sera overnight. The following day filters were again washed and subsequently blocked preceding incubation in anti-human IgG-AP. Filters were once again washed and detected using 5-bromo-4-chloro-3-indolyl phosphate/nitroblue tetrazolium.

Developed filters were scanned into high quality digital format. Images were then aligned to a 9×8 grid pattern and automatically screened for the density of each spot. Background correction was done by calculating the average density of pixels immediately around each spot and subtracting that average from spot density. Data was normalized to the average of IgG replicates on each filter.

Cloning of λ pTripleX CT phage: Image clones of 27 different CT antigens obtained from ATCC were plasmid purified and PCR amplified with primers designed to introduce the 5′ EcoRI or MfeI and 3′ XhoI or SalI. Amplified products were gel purified and ligated into pTripleX phage arms and subsequently packaged using Gigapack III gold packaging extract. Product phages were PCR amplified and re-sequenced to confirm identity.

Expression of desired mRNA was confirmed from phage-infected E. coli by RTPCR.

Tumor and Tissue PCR/RTPCR: CTAs of interest were tested by PCR for testis exclusivity in cDNA libraries from human liver, lung, testis, colon, bladder, prostate, heart, brain, kidney, placenta, skeletal muscle, spleen, and thymus. PCR was also used to screen metastatic tissue cDNAs as well as prostate cancer and normal prostate epithelial cell lines.

SSX-2 peptide-specific cytotoxicity assay: Peripheral blood mononuclear cells from an HLA-A2-expressing patient with prostate cancer were cultured in the presence of known HLA-A2-specific nonamer peptide epitopes derived from the influenza matrix protein (flu, GILGFVFTL, SEQ ID NO:7, positive control) or SSX-2 (RLQGISPKI, SEQ ID NO:8) (Wagner et al., 2003, Cancer Immunol. 3:18-32). After five to six weekly restimulations in vitro, lactate dehydrogenase (LDH) cytotoxicity assay was conducted to evaluate cells for peptide-specific cytotoxicity using HLA-A2-expressing T2 cells pulsed with relevant or irrelevant peptides.

Results

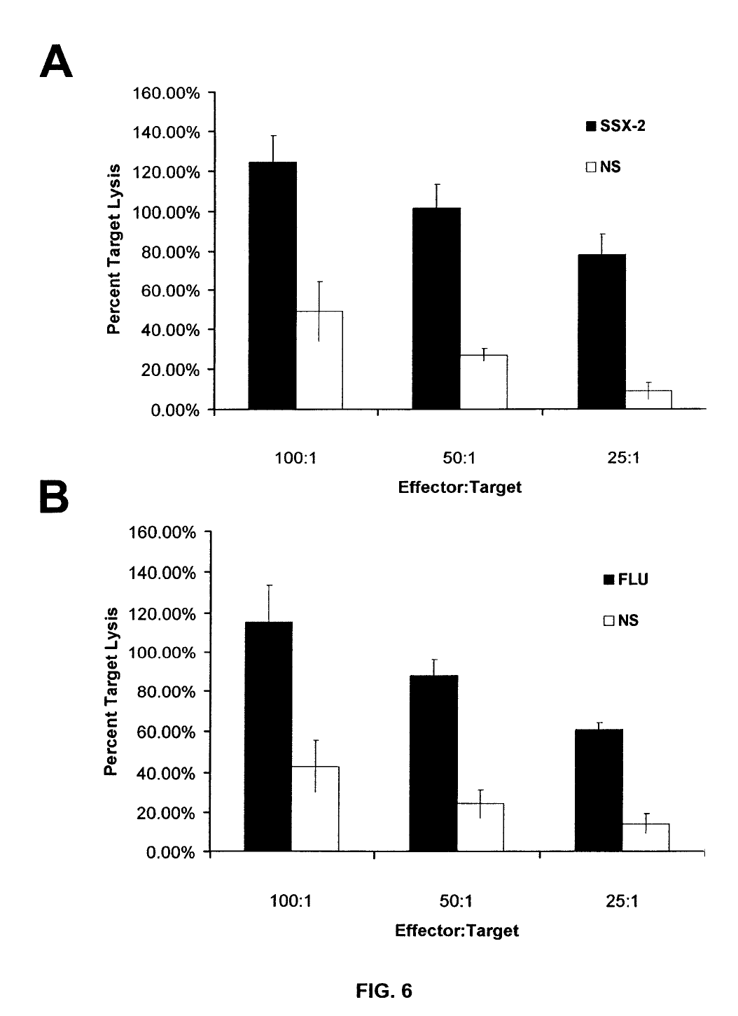

As shown in FIG. 1, sequence confirmed phage showed mRNA expression by RTPCR. Phage-infected XL-1 blue strain of E. coli were processed for total RNA. RTPCR analysis was conducted on these samples using gene specific primers. Positive control was sequenced phage DNA, negative control was pre-boiled RTPCR reactions, inactivating reverse transcriptase activity. Data is representative of results obtained.

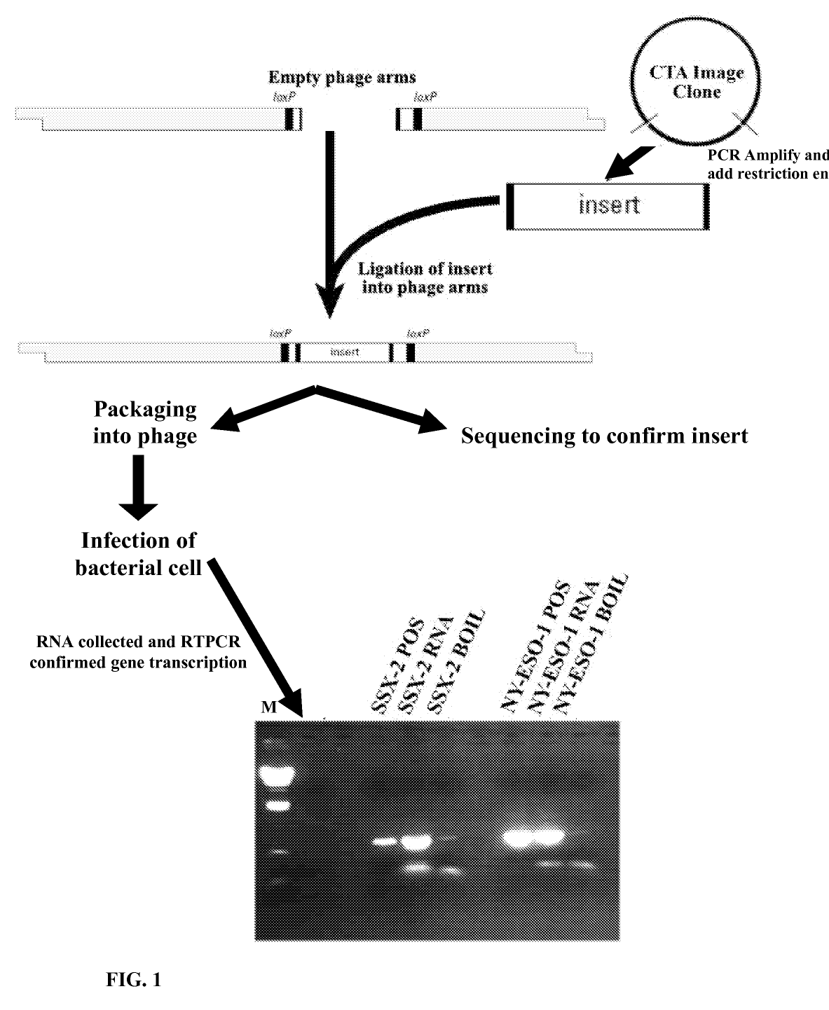

As shown in FIG. 2, high throughput immunoblot (HTI) methodology provides an efficient means of quantitatively mass screening sera for reactivity to multiple antigens. Phage at 10,000 pfu were spotted onto Top Agarose Omniwell plates utilizing a Biomek FX liquid handling robot optimizing for multiple replicates in a mass screening. After incubation with IPTG-nitrocellulose filters and subsequent detection, filters were scanned into digital images. These images were aligned on a 9×8 grid pattern and an automated process locates spots on grid and calculates densitometry. Densitometric data was then normalized for each sera establishing an empty phage construct as “0” and a phage encoding human IgG as “1.0”. The line shows the mean plus three standard deviations of the normal control population.

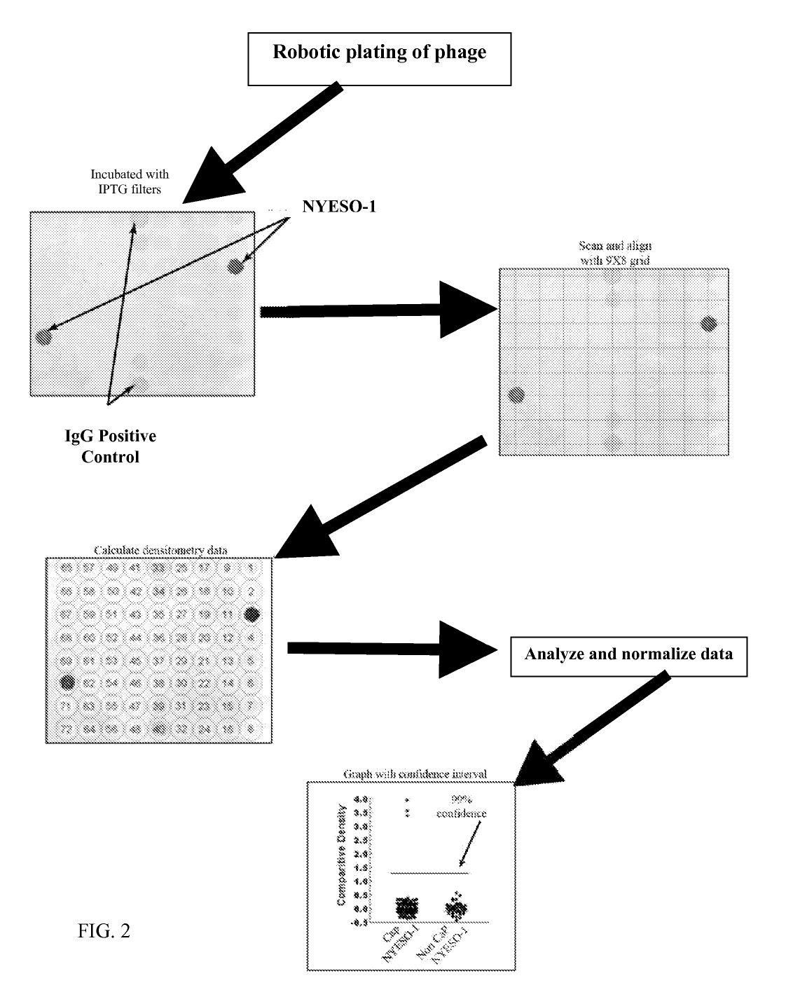

As shown in FIG. 3, HTI identified significant IgG responses to at least 2 (SSX-2 and NY-ESO-1) of 29 known CTAs in sera from prostate cancer patients. The HTI data for the 29 analyzed antigens and a negative control “empty” λ-phage was graphed along with 99% confidence intervals for each antigen set. Each data point indicates IgG reactivity in a single patient's serum. All data points were graphed and each antigen is represented by IgG response data among the cancer and normal populations. Data were normalized for each sera establishing an empty phage construct as “0” and a phage encoding human IgG as “1.0”. The line denoting the 99% confidence interval shows the mean plus three standard deviations of the normal control population.

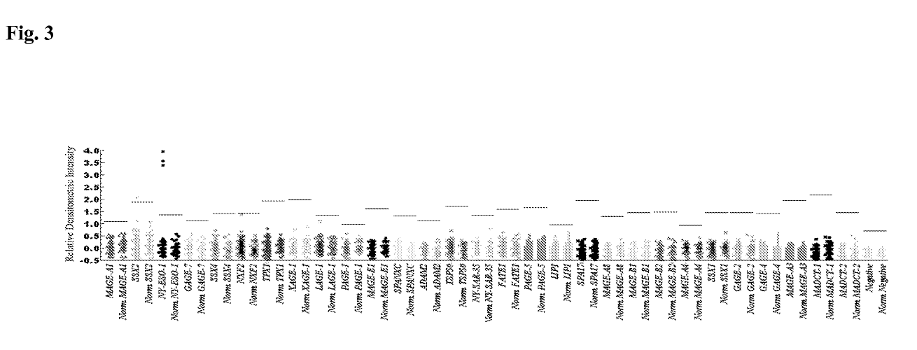

As shown in FIG. 4A, SSX-2 showed testis-specific expression among normal tissues. Tissue cDNA were tested by PCR for expression of SSX-2 using gene specific primers. The negative control (NEG) used water in place of tissue cDNA and the positive control (POS) was phage encoding SSX-2. Markers (M) were used to calculate size. Similar experiment was conducted for NYESO-1 and testis-specific expression among normal tissues was observed (data not shown).

As shown in FIG. 4B and FIG. 4C, we found SSX-2 expression in human prostate cancer metastatic sites and prostate cancer cell lines. In FIG. 4B, cDNA from prostate cancer metastatic tissue from seven different patients was evaluated for SSX-2 gene transcription by PCR using gene-specific primers. Lane 1: small intestine metastasis; Lane 2-4: lymph node metastases; Lane 5-7: bone metastases. Positive control (POS) used SSX-2 phage and the negative control (NEG) used water. Markers (M) were used to calculate size. SSX-2 expression was detected in the metastatic tissues from patients 1 (intestine) and 2 (lymph node). In FIG. 4C, RNA from prostate epithelial and cancer cell lines were evaluated by RTPCR for SSX-2 gene transcription. Epithelial cell lines were in order from 1-5: PrEC1, PrEC2, PrEC3, PrEC4, and PZ-HPV7. Cancer cell lines were in order from 6-11: SWPC1, SWPC2, SWNPC2, LAPC4, MDAPca2b, and MDAPca2a. The positive control (POS) used SSX-2 phage. Markers (M) were used to calculate size. While none of the normal prostate epithelial cell lines expressed SSX-2, two of the prostate cancer cell lines did.

The inventors also conducted immunohistochemical staining of LNCap prostate cancer cell line with a antibody specific for SSX-2 and found that the cell line expressed SSX-2 (data not shown).

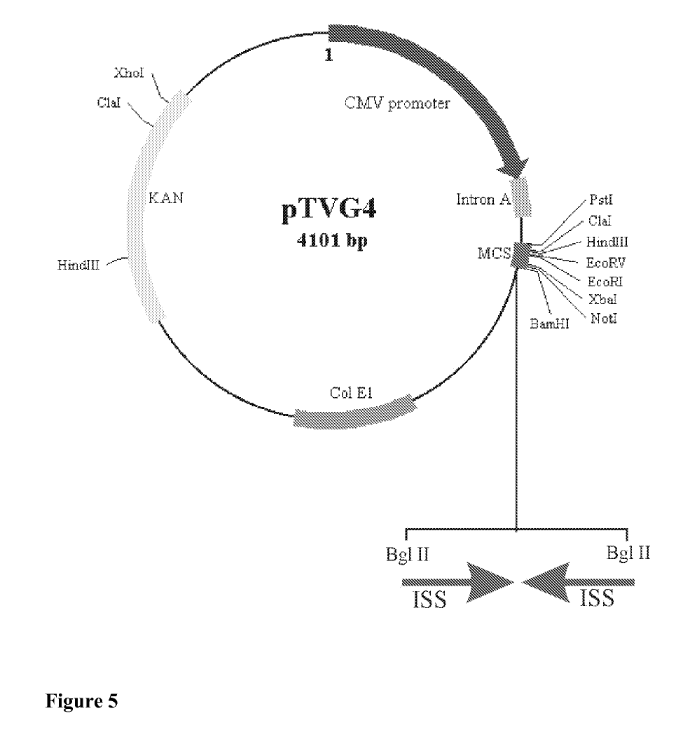

As shown in FIG. 6, cytotoxic T cells specific for an HLA-A2 peptide epitope presented by SSX-2 were identified the peripheral blood of a patient with prostate cancer. Peripheral blood mononuclear cells from an HLA-A2-expressing patient with prostate cancer were cultured in the presence of known HLA-A2-specific nonamer peptide epitopes derived from the influenza matrix protein (flu, GILGFVFTL, SEQ ID NO:7, positive control) or SSX-2 (RLQGISPKI, SEQ ID NO:8) (Wagner et al., 2003, Cancer Immunol. 3:18-32). After five to six weekly restimulations in vitro, cells were evaluated for peptide-specific cytotoxicity using HLA-A2-expressing T2 cells pulsed with relevant or irrelevant peptides. FIG. 6 shows the results of lactate dehydrogenase (LDH) cytotoxicity assays with these two cultures (Panel A, SSX-2 peptide-specific cultures; Panel B, flu peptide-specific cultures). Bars show the average and standard deviation of percent LDH specific release from three replicates for each effector-to-target ratio. NS (non-specific) represents irrelevant peptide-pulsed group. As shown in FIG. 6, cytotoxicity was observed for T2 cells pulsed with the relevant SSX-2 or flu peptide, but not T2 cells pulsed with an irrelevant HLA-A2-binding peptide.

### EXAMPLE 2 (PROPHETIC)

### Rats Immunized with Human SSX-2 Protein Develop Cross-Reactive Immunity to Rat SSX-2 and Immunization with Human SSX-2 Protein Elicits Helper T Cells and Antibodies

Lewis rats are immunized with human SSX-2 protein in Freund's adjuvant and a proliferative T cell and antibody response specific for human SSX-2 is expected to develop. Splenocytes from immunized rats are prepared by gradient centrifugation (Histopaque, Sigma). Human SSX-2 and ovalbumin, as a negative control protein, are used as test antigens for primed T cell responses in a ³H-thymidine incorporation assay. Sera from an immunized rat are used to evaluatc SSX-2-specific antibody responses in a Western blot experiment. A cross-reactive antibody response in these animals will recognize the rat SSX-2 protein homologue and this response will not be seen in control animals.

### EXAMPLE 3 (PROPHETIC)

### Rats Immunized with Peptides Derived from Human SSX-2 Develop SSX-2-Specific Immune Responses

Male Lewis rats are immunized three times at 14-day intervals with 50 μg of a distinct short peptide (e.g., a 15-mer) derived from the amino acid sequence of human SSX-2 using Freund's adjuvant. An irrelevant short peptide (e.g., a 15-mer) can be used as a negative control. Immunization with the human SSX-2 short peptide is expected to elicit a peptide-specific and human SSX-2 protein-specific T cell proliferative response. In addition, an antibody response specific for human SSX-2 protein is expected in the same immunized rats. Similarly, a T-cell proliferative response specific for a peptide encompassing the corresponding rat SSX-2 homologue is expected following immunization with either the human- or rat-specific SSX-2 peptide.

### EXAMPLE 4 (PROPHETIC)

### DNA Vaccines Encoding Either Human or Rat SSX-2 Elicit Antigen-specific Cellular Immunity in Rodent and Human in vitro Models

1. Preparation of cDNA encoding human SSX-2, rat SSX-2 and control antigen.

mRNA encoding the green fluorescent protein (GFP), human SSX-2, or the rat SSX-2 homologue is prepared from total RNA from cell lines or tissues expressing the gene of interest. The cDNA encoding the antigen is then reverse transcribed using standard methods from the mRNA. Alternatively, the cDNA is directly available as a commercially available IMAGE clone. The sizes of the cDNA products are then confirmed on agarose gel. These cDNA products are used for the construction of the plasmid vaccines described below.

2. Construction of pTVG4.

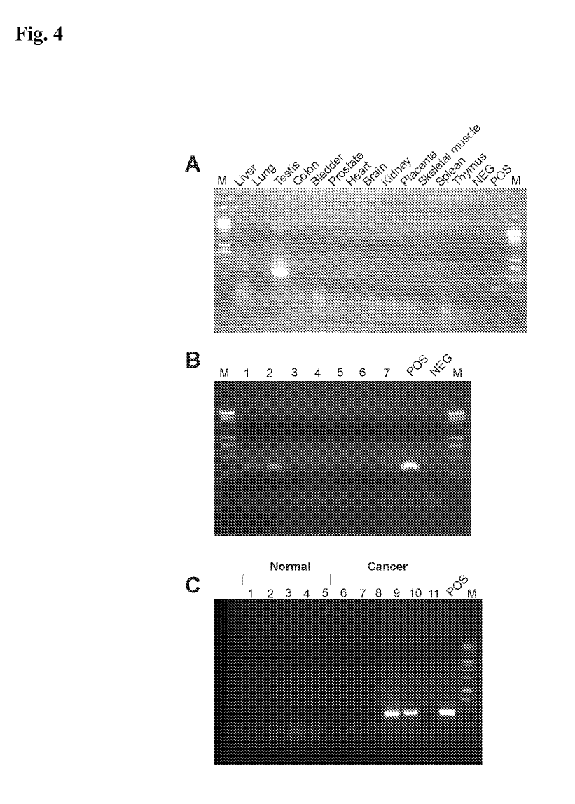

Plasmid DNA expression vectors have been developed for use in human vaccines. Shown in FIG. 5 is a plasmid map for the pTVG4 vector as constructed for rat immunization experiments and human trials. Into this construct is inserted the coding sequence for the human SSX-2 gene or rat homologue, to create the immunization vectors pTVG-HSSX2 and pTVG-RSSX2 (see below).

The plasmid vector pNGVL3 can be obtained from the National Gene Vector Laboratory at the University of Michigan (Dr. Robert Gerard). This vector, similar to the pCDNA3.1 expression vector, drives transcription from the CMV promoter, but also includes the CMV intron A sequence to enhance protein expression (Lee et al., 1997, Mol. Cells 7:495 501). The vector also contains a multi-cloning site, and does not express a eukaryotic antibiotic resistance gene, such that the only protein expression expected in a eukaryotic system is the one driven from the CMV promoter, unlike the pCDNA vector. To this vector is added 2 copies of a 36-bp immunostimulatory (ISS) fragment containing the 5′ -GTCGTT-3′ motif previously identified (Hartmann et al., 2000, J. Immunol. 164:1617-24), to create the vector pTVG4 (FIG. 5). The coding sequence for human SSX-2 or the rat homologue is cloned into this vector, and expression of SSX-2 can be confirmed by in vitro expression studies and immunoblot analysis. These constructs, named pTVG-HSSX2 and pTVG-RSSX2, respectively, are used for the immunization studies.

3. Construction of pTVG-HSSX2 for human clinical trials.

The cDNA coding sequence for human SSX-2 is cloned into this vector to produce the construct pTVG-HSSX2. This is the construct to be used for clinical trials. Transient transfection of Chinese Hamster Ovary (CHO) cells or human dendritic cells followed by capture ELISA can be used to confirm that human SSX-2 is expressed in vitro. In addition, human SSX-2 expression may be confirmed by an in vivo study as well. Specifically, 500 μg of either pTVG4 or pTVG-HSSX2 plasmid DNA is administered to male Lewis rats by direct administration to the right external iliac artery. Sera are obtained 5 days after administration and animals are euthanized after ten days. Sera are then evaluated for SSX-2 protein concentration by capture ELISA, and hind limb muscle biopsies are stained immunohistochemically for SSX-2 expression.

4. Plasmid DNA stimulates antigen-specific T cell responses in vitro.

Cryopreserved peripheral blood mononuclear cells (PBMC) from patients with metastatic prostate cancer are thawed and re-suspended at 10⁶ cells/ml in RPMI medium (Gibco BRL, Rockville, Md.) supplemented with 10 mM L-glutamine, 2% penicillin/streptomycin, 50 μM β-mercaptoethanol and 10% human AB serum (Valley Biomedical, Winchester, Va.), as well as 2 μg/ml of a plasmid DNA construct encoding the rat SSX-2 protein (pTVG-RSSX2, containing the cDNA for rat SSX-2 in place of the human SSX-2 cDNA), the human SSX-2 protein (PTVG-HSSX2), or the vector control plasmid containing no cDNA insert, pTVG4. Proliferating lymphocytes are twice re-stimulated at 7-10 day intervals with irradiated (3300 cGy) autologous PBMC and pulsed with DNA at 2 μg/ml. Media are exchanged every 3-4 days after stimulation with the T cell medium described above containing 10 U/ml rhIL-2 (Chiron, Emeryville, Calif.). After 3 in vitro stimulations, cultured T cells are assayed for proliferative T cell responses to SSX-2 (2 μg/ml), peptides derived from SSX-2, PHA (2.5 μg/ml as a positive control), or no antigen, using irradiated autologous PBMC as antigen presenting cells, as previously described (McNeel et al., 2001, Cancer Res. 61:5161 5167). It is expected that human SSX-2-specific proliferative T cell responses be detected after 3 in vitro stimulations with the vaccine DNA construct encoding human or rat SSX-2.

Similarly, PBMC from patients with metastatic prostate cancer are allowed to adhere to tissue culture flasks, and adherent cells are cultured in Aim V medium (Gibco) supplemented with GM-CSF and IL-4. After 6 days, the cultured dendritic cells are pulsed with 10 μg/ml pTVG4 or pTVG-RSSX2 and 10 U/ml TNFα. Fresh PBMC are then added at a 10:1 effector:APC ratio, cultured for 7 days, and then re-stimulated with DNA-pulsed dendritic cells, as above. After an additional week, cells are assayed for antigen-specific T cell proliferation. It is expected that both CD4 and CD8 T cells specific for human SSX-2 will be detected following stimulation with DNA encoding human or rat SSX-2, but not the control plasmid.

### EXAMPLE 5 (PROPHETIC)

### Rats Immunized with pTVG-HSSX2 Develop SSX-2-Specific Cellular Immunity

2-3 month-old male Lewis rats are immunized intradermally with 100 μg of plasmid DNA and 5 μg rat GM-CSF protein as an adjuvant. Immunization is repeated every 2 weeks for up to six total immunizations, and animals are sacrificed two weeks after the boost. Rodents immunized with pTVG-HSSX2 or pTVG-RSSX2, but not the pTVG4 control vector, are expected to develop a proliferative CD4 and CD8-T cell response to human SSX-2 and the rat homologue. In addition, animals receiving pTVG-HSSX2 as a vaccine are expected to develop a detectable antibody response to SSX-2. Moreover, cytotoxic T cell (CTL) responses specific for SSX-2 are expected after in vitro stimulation and to lyse a syngeneic Lewis rat pancreatic cancer cell line, DSL, engineered to express human SSX-2 or the rat homologue in pTVG-HSSX2- or pTVG-RSSX2-immunized animals. CTL responses specific for SSX-2 are not expected in any control animals.

### EXAMPLE 6 (PROPHETIC)

### Administration of Plasmid DNA Encoding SSX-2 Intraarterially Leads to High Levels of Antigen Expression

Male Lewis rats receive 500 μg of control pTVG4 plasmid or plasmid expressing human SSX-2 (pTVG-HSSX2) or the rat homologue (pTVG-RSSX2), by either intradermal administration (n=3) or intraarterial administration into the external iliac artery (n=2). Sera are obtained on day 5 from each animal and analyzed by quantitative capture ELISA for serum human SSX-2 concentrations. It is expected that administration of plasmid DNA encoding SSX-2 intraarterially leads to high levels of antigen expression.

### EXAMPLE 7 (PROPHETIC)

### Plasmid DNA Used for Immunization can be Detected at the Site of Immunization at Least 2 Weeks after Vaccination

Lewis rats are immunized intradermally on days 1 and 15 with 50 μg of plasmid DNA. Two weeks after the second immunization, animals are sacrificed and biopsies taken from the site of immunization. Samples are treated overnight with proteinase K, and the nucleic acids precipitated with ethanol. Detection of plasmid DNA is done by PCR using plasmid-specific primers. It is expected that plasmid DNA used for immunization can be detected at the site of immunization at least 2 weeks after vaccination.

### EXAMPLE 8 (PROPHETIC)

### Rat Immunization with a Plasmid DNA Vaccine

Groups of six 2-month old male Lewis rats are immunized twice at 2-week intervals, using 50, 100, or 500 μg of pTVG-HSSX2 or 500 μg control vector pTVG4 with 5 μg rat GM-CSF as a vaccine adjuvant. Immunizations are performed intradermally in a 100 μl total volume of normal saline. Other groups of six male Lewis rats receive 50, 100, 500 or 1500 μg pTVG-HSSX2 DNA, or 1500 μg control pTVG4, suspended in 10 ml of normal saline, and administered once by rapid injection intraarterially in the hind limbs, as previously described (Budker, et al., 1998, Gene Ther. 5:272 276). Blood is drawn from the tail vein 48 hours and 5 days after each immunization, or intraarterial administration, for serum SSX-2 levels. Animals are sacrificed two weeks after the second immunization or intraarterial administration with collection of blood, spleens, prostate tissue biopsy, and hind limb muscle biopsy. This study is performed in two sets at different times with nine groups of three animals per group. Immunological responses are assessed, evaluating SSX-2-specific antibody responses, cytokine-secreting T cell responses by ELISPOT, CD4 and CD8 antigen-specific T cell proliferation, and assays of antigen-specific cytotoxic T cells. Direct immunohistochemical evaluation of SSX-2 expression and inflammation is with the hind limb muscle biopsies. Peptide-specific T cells are also assessed as described above, to determine the peptide epitopes presented by DNA administered by the intraarterial route compared with the intradermal route of delivery.

### EXAMPLE 9 (PROPHETIC)

### Administration of Plasmid DNA Encoding SSX-2 Intravenally Leads to High Level of Antigen Expression in Hepatocytes

The plasmid vectors of the present invention may be delivered to the patient in need thereof intravascularly. Intravascular delivery may be by direct peripheral artery or venous delivery, or by visceral arterial or venous infusion, such as by direct delivery to the hepatic artery, using well established methods known to those with ordinary skills in the art. Intravenous delivery in rats may be by tail vein injection, when appropriate. This delivery leads to a high level of antigen expression in hepatocytes. Expression of the antigen in liver, a tissue more rich with antigen-presenting cells, can lead to a more pronounced Th1 /CTL response than expression in muscle tissue.

### EXAMPLE 10 (PROPHETIC)

### Immunization of Male Lewis Rats with Plasmid DNA Vaccines Elicits T Cell Immune Responses Cross-reactive with the Xenoantigen

2-3 month-old male Lewis rats are immunized intradermally with 100 μg pTVG4, pTVG-HSSX2, or pTVG-RSSX2 and 5 μg rat GM-CSF (mGM-CSF) adjuvant at least twice, as described above. 14 days after the final immunization, animals are euthanized and assessed for T cell proliferative responses after culture with media only (no antigen), 2 μg/ml purified human SSX-2 protein, to a pool of several short peptides (e.g., 15-mer) derived from (and specific for) human SSX-2, a pool of several short peptides (e.g., 15-mer) derived from (and specific for) rat SSX-2, or PHA as a positive control. The control treated animal are not expected to develop human SSX-2 protein or human SSX-2-derived peptide-specific responses. The animals treated with pTVG-HSSX2 and the animals treated with the peptides are expected to develop human SSX-2-specific CD4 and CD8 T cells. The animal treated with pTVG-RSSX2 are expected to develop rat SSX-2 peptide-specific T cell responses, as well as a response to the human SSX-2 protein and human SSX-2-specific peptides. In addition, some animals immunized with pTVG-HSSX2, but not pTVG4, may develop a cross-reactive immune response recognizing rat SSX-2.

### EXAMPLE 11 (PROPHETIC)

### Xenoantigen Priming Followed by Boosting with Autoantigen or Xenoantigen

The DNA vaccines of the present invention can be used in a prime-boost strategy to induce robust and long-lasting immune response to SSX-2.

1. Vectors and Immunization Procedures.

A plasmid vector pTVG4, a derivative of pNGVL3 (a plasmid designed for DNA immunization and obtained from the National Gene Vector Laboratories at the University of Michigan), is constructed. The plasmid pTVG-HSSX2 contains the cDNA for human SSX-2 downstream of a CMV promoter, and the plasmid pTVG-RSSX2 contains the cDNA for the rat SSX-2 homologue downstream of the CMV promoter in place of the human SSX-2 cDNA, both within the pTVG4 vector.

Groups of six 2-3 month old male Lewis rats are immunized with 100 μg of plasmid DNA encoding human SSX-2 (pTVG-HSSX2), rat SSX-2 (pTVG-RSSX2), or the parental control plasmid pTVG4. Immunization is performed intradermally in the ear pinna, with 5 μg of rat GM-CSF as a vaccine adjuvant. After 2 weeks, immunizations are boosted with 100 μg of plasmid DNA encoding the same protein or the homologue. Control animals receive booster immunization with the vector alone. Animals are sacrificed two weeks after the booster immunization with collection of sera and spleens.

A second set of experiments explore different prime-boost strategies. Specifically, groups of 2-3 month-old male Lewis rats receive 500 μg of either pTVG-HSSX2 or pTVG-RSSX2 by rapid intraarterial delivery as described above. Two weeks later, animals receive either the human SSX-2- or rat SSX-2-encoding plasmid administered intradermally. A control group receives the pTVG4 plasmid by both delivery methods. Animals are sacrificed two weeks after the booster immunization with collection of blood and spleens for immunological analysis, and prostate and hind limb muscle tissue biopsies for immunohistochemical analysis.

Immunological analysis is performed as described above, with analysis of the peptide-specific T cell epitopes recognized. These experiments test not only the ability of a xenoantigen to prime a response to the native antigen, but also determine if an intradermal booster immunization is effective in augmenting the immunity elicited by intraarterial administration of plasmid DNA.

2. Immunological evaluation.

Humoral immune responses to human SSX-2 and rat SSX-2 are quantitatively evaluated in experimental animals using an indirect ELISA method. Specifically, the cDNAs for human SSX-2 and rat SSX-2 are cloned into the pCDNA4/HisMax expression construct (Invitrogen) in such a fashion to generate a polyhistidine epitope tag at the amino terminus of each gene product. Capture ELISAs are performed as previously described (McNeel et al., 2001, Proc. Amer. Assoc. Cancer Res. 42:156) using a monoclonal antibody specific for the polyhistidine tag to capture either the human or rat antigen from CHO cells transfected to express these proteins. Rat sera are then used to screen for anti-SSX-2 specific responses, with antibodies detected by anti-rat Ig secondary antibodies, again as previously described (McNeel, et al., 2000, J. Urol. 164:1825 1829). IgG subsets are determined using subset-specific secondary antibodies.

Second, T cell responses specific for human SSX-2 or rat SSX-2 are evaluated in individual experimental animals by both T cell proliferation and ELISPOT. T cell proliferation is assessed by standard ³H-thymidine incorporation assays or by flow cytometric analysis of BrdU incorporation using a BD Pharmingen kit BD Biosciences (San Diego, Calif.), using either purified SSX-2 protein, peptides derived from human SSX-2 or the rat homologue, or stable transfectants of the syngeneic Lewis rat DSL pancreatic cell line expressing either human or rat SSX-2. ELISPOT is used to assess both IFNγ and IL-4 cytokine secretion in response to antigen stimulation using the same purified protein, peptides, or SSX-2-expressing DSL cell lines described. These ELISPOT methods have been described (McNeel et al., 2001, Proc. Amer. Assn. Cancer Res., 42:277; Zou et al., 1999, J. Neuroimmunol. 94:109 121). Specifically, sterile 96-well ELISPOT filter plates (Millipore) are coated with 5 μg/ml capture antibody (IFNγ clone DB1, Harlan; IL-4 clone OX-81, Pharmingen) overnight. About 10⁴ splenocytes from immunized animals are then co-cultured in replicate wells with SSX-2-expressing irradiated (120 Gy) DSL cell lines, a control transfected DSL cell line, or 40 ng/ml PMA+0.4 μg/ml ionomycin as a positive control. After 48 hours, plates are washed, sequentially probed with secondary antibody (biotinylated anti-IL-4, RDI; polyclonal rabbit anti-rat IFNγ, RDI) and tertiary reagent (streptavidin-conjugated alkaline phosphatase, BioRad; or anti-rabbit IgG conjugated to alkaline phosphatase, Sigma), and then developed with BCIP/NZT substrate (BioRad). Spots are then enumerated with determination of the responder cell frequency per 10,000 splenocytes. A Student's t test is used to compare the number of spots from replicate antigen-stimulated wells with control wells, and between immunized and control animals, to detect significant differences in T cell frequencies.

Finally, cytotoxicity is assessed by an in vitro assay. For the in vitro assays, stable transfectants of the DSL cell line expressing human or rat SSX-2 are used as target cells in standard cytotoxicity assays using effector splenocytes in several effector-to-target ratios. Cytotoxicity is assessed by flow cytometry using propidium iodide staining of fluorescently labeled target cells [Molecular Targets], or by release of LDH (Cytox 96 Assay kit, Promega Corporation).

3. Statistical analysis.

In the first set of experiments, there are five primary treatment groups: control and four prime-boost experimental groups. In the second set of experiments, there are five primary treatment groups: two controls and three experimental groups. A group size of 6 animals (30 rats for the first initial experiments and 24 for the initial second experiments) provides 90% power to detect large changes detectable by semi-quantitative proliferative assays or quantitative continuous-variable ELISPOT assays.

Results from these studies are expected to show that plasmid DNA booster immunization with the autologous rat SSX-2 primed with the human SSX-2 xenoantigen is more effective than immunization with the xenoantigen alone.

### EXAMPLE 12 (PROPHETIC)

### Multiple Immunizations

Multiple immunizations are also used. Repeated immunization with either the autologous rat SSX-2 vaccine or the human SSX-2 vaccine, or in alternating schedules, may be used to produce a strong autoantigen-specific immune response. This is one of the potential advantages of plasmid DNA vaccines over other types of vaccines, such as viral vaccines, in which repeated immunizations are not possible.

### EXAMPLE 13 (PROPHETIC)

### Clinical Trials

Clinical trials are conducted to test the ability of a plasmid DNA construct encoding human SSX-2 to elicit SSX-2-specific CD8 T cell responses in subjects with high-risk prostate cancer. pTVG-HSSX2, a derivative of the pNGVL3 vector obtained from the National Gene Vector Laboratories, is used, and clinical grade DNA is manufactured for clinical trials.

### EXAMPLE 14 (PROPHETIC)

### Detecting and Monitoring Antigen-Specific Immune Responses to SSX-2 in Patients with Prostate Cancer

1. SSX-2-specific MHC class I-restricted T cell responses can be detected in patients with prostate cancer by ELISPOT.

A plurality of oligopeptides (e.g., 9-15 mer) are chosen from the amino acid sequence of SSX-2 based on computer modeling and sequence as likely HLA-A2 binding epitopes, and specific for SSX-2. These peptides are constructed, ranked for HLA-A2 binding by an in vitro T2 assay, and evaluated as stimulator antigens in an IFNγ ELISPOT assay after one in vitro stimulation using the PBMC from a HLA-A2-expressing patient with stage D0 prostate cancer. Positive controls include an oligopeptide (e.g. a 9-15 mer) HLA-A2 epitope from influenza and tetanus toxoid. Individual HLA-A2-binding epitopes from SSX-2 are expected to elicit IFNγ-secreting lymphocytes, and these can be detected in some patients with prostate cancer. Additionally, some HLA-A2-specific epitopes derived from SSX-2 are already known (Wagner et al., 2003, Cancer Immunol. 3:18-32).

Using this methodology and evaluating multiple HLA-A2-positive patients and controls, SSX-2-specific HLA-A2 epitopes with high affinity for HLA-A2 can be identified, for which peptide-specific T cells can be identified in subjects with prostate cancer. This approach has been successfully used for PAP (McNeel et al., Identification of PAP-specific MHC class I peptide epitopes by screening patients with prostate cancer by IFN-gamma ELISPOT. (2001) Proc. Amer. Assn. Cancer Res., 42:277). Peptide-specific lines for these identified peptides can be cultured from HLA-A2-positive patients with prostate cancer, with the demonstration of peptide specificity. As an example, an oligopeptide derived from SSX-2 can be used as a stimulator antigen in short-term T cell cultures stimulated with autologous GM-CSF and IL-4 generated dendritic cells from an HLA-A2-positive subject with prostate cancer. After several weekly in vitro stimulations with irradiated autologous peptide-pulsed PBMC, cultures are found to be predominantly CD8 by flow cytometric phenotype analysis and then evaluated for peptide-specific CTL activity.

2. Dimeric human HLA-A2:Ig can be used to identify peptide-specific CD8 T cells specific for SSX-2.

The ability of soluble MHC molecules fused to IgG to identify and quantify peptide-specific T cells is known (Lebowitz et al., Soluble, high-affinity dimers of T-cell receptors and class II major histocompatibility complexes: biochemical probes for analysis and modulation of immune responses. (1999) Cell Immunol, 192: 175 184). This technology, similar to the tetramer technology, can be successfully applied in multiple systems to identify, activate, or suppress the activity of peptide-specific T cells

Dimeric human HLA-A2:Ig loaded with an oligopeptide described above is used to identify peptide-specific CD8 T cells cultured in vitro.

### EXAMPLE 15 (PROPHETIC)

### Immunization with pTVG-HSSX2 or pTVG-RSSX2 Elicits Therapeutic Anti-Prostate Tumor Response

Male Copenhagen rats are treated on day 0 with 10⁴ Mat-Lu cells (transplantable prostate tumor cells) implanted subcutaneously in Matrigel (BD Pharmingen). If Mat-Lu cells do not express SSX-2 per se, they will be transfected with an expression vector to express human SSX-2 or the rat SSX-2 homologue before implantation. Animals are treated in random fashion, to not bias tumor implantation, and then subsequently assigned to treatment groups. Animals are then immunized on days 1 and 15 with 100 μg pTVG-HSSX2 (n=6), pTVG-RSSX2 (n=6), or PBS (n=3only. Bidimensional tumor measurements are obtained beginning on day 23. The mean tumor volume of pTVG-HSSX2- or pTVG-RSSX2-immunized animals is expected to be smaller than that of control animals (PBS immunized). This shows that pTVG-HSSX2 and pTVG-RSSX2 are able to elicit therapeutic anti-tumor response in vivo.

### EXAMPLE 16 (PROPHETIC)

### Immunization with pTVG-HSSX2 or pTVG-RSSX2 Elicits Protective Anti-Prostate Tumor Response

Male Copenhagen rats are immunized four times with pTVG4 (n=10), pTVG-HSSX2 (n=10), or pTVG-RSSX2 (n=10). Two weeks after the last immunization, animals are challenged with 10⁶ Mat-Lu cells (may need to be transfected with an expression vector to express human SSX-2 or the rat SSX-2 homologue, see example 15) administered subcutaneously. A Kaplan Meier analysis is conducted with events determined once bidimensional tumor sizes measured 10 cm³ in size. This analysis is expected to show that immunization with pTVG-HSSX2 or pTVG-RSSX2 is able to elicit protective anti-tumor response in vivo.

### EXAMPLE 17 (PROPHETIC)

### Immunization with pTVG-HSSX2 or pTVG-RSSX2 elicits Cytotoxic-T-lymphocyte (CTL) Response

Male Copenhagen rats receive 10⁴ MatLu cells (may need to be transfected with an expression vector to express human SSX-2 or the rat SSX-2 homologue, see example 15) administered subcutaneously on day 1, followed by two intradermal immunizations (on days 2 and 16) with 100 μg of control pTVG4 plasmid (n=6), pTVG-HSSX2 (n=6) or pTVG-RS SX2 (n=6) Animals are sacrificed on day 45, and splenocytes pooled from animals per group. Splenocytes are stimulated in vitro with irradiated MatLu cells (120 Gy) in a 10:1 ratio for 6 days with the addition of 10 U/ml rIL-2 on day 4. CTL activity to MatLu target cells is detected by lactate dehydrogenase (LDH) release (Cytox 96 kit, Promega) on day 7. The mean and standard deviation of % specific lysis for triplicate samples against the Mat-Lu targets using effector cells from animals immunized with pTVG4, pTVG-HSSX2, or pTVG-RSSX2 are expected to show that immunization with pTVG-HSSX2 or pTVG-RSSX2, but not pTVG4, elicits SSX-2-specific CTL.

The present invention is not intended to be limited to the foregoing examples, but encompasses all such modifications and variations as come within the scope of the appended claims.

We claim:

1. A method for inducing an immune reaction to SSX-2 in a mammal having prostate cancer, comprising administering to the mammal an effective amount of a recombinant DNA construct comprising a polynucleotide sequence encoding SSX-2 or an immunogenic fragment thereof operatively linked to a transcriptional regulatory element, whereby the mammal develops immune reaction against SSX-2.

2. The method of claim 1, wherein the polynucleotide sequence encodes a human SSX-2.

3. The method of claim 1, wherein the polynucleotide sequence encodes a rat or mouse homologue of SSX-2.

4. The method of claim 1, wherein the recombinant DNA construct is administered to the mammal intradermally, intramuscularly, or intravascularly.

5. The method of claim 1, wherein the mammal is human.

6. The method of claim 1, wherein cellular immune reaction against cells expressing SSX-2 is induced.

7. The method of claim 6, wherein both humoral and cellular immune reactions against SSX-2 are induced.

8. A method for inducing immune reaction to SSX-2 in a mammal having prostate cancer, comprising administering to the mammal an effective amount of a first recombinant DNA construct comprising a first polynucleotide sequence encoding a first SSX-2 polypeptide or an immunogenic fragment thereof operatively linked to a transcriptional regulatory element; and administering to the mammal an effective amount of a second recombinant DNA construct comprising a second polynucleotide sequence encoding a second SSX-2 polypeptide or an immunogenic fragment thereof operatively linked to a transcriptional regulatory element; wherein the first polynucleotide sequence and the second polynucleotide molecule originate from two different animal species, whereby an immune reaction against SSX-2 is induced in the mammal.

9. The method of claim 8, wherein the first polynucleotide sequence originates from an animal species other than the mammal, and the second polynucleotide sequence originates from the same animal species as the mammal.

10. The method of claim 8, wherein the mammal is a human, and the first polynucleotide sequence encoding the rat or mouse homologue of SSX-2.

11. The method of claim 8, wherein the second polynucleotide sequence originates from the same animal species as the mammal, and the first polynucleotide sequence encodes an SSX-2 polypeptide that shares at least 85% homology to the first SSX-2 polypeptide.

12. The method of claim 11, wherein the first polynucleotide sequence encodes an SSX-2 polypeptide that shares at least 88% homology to the first SSX-2 polypeptide.

13. The method of claim 11, wherein the first polynucleotide sequence encodes an SSX-2 polypeptide that shares at least 90% homology to the first SSX-2 polypeptide.

14. The method of claim 11, wherein the first polynucleotide sequence encodes an SSX-2 polypeptide that shares at least 95% homology to the first SSX-2 polypeptide.

15. The method of claim 11, wherein the first polynucleotide sequence encodes an SSX-2 polypeptide that shares at least 98% homology to the first SSX-2 polypeptide.

16. The method of claim 11, wherein the first and the second recombinant DNA constructs are administered to the mammal intradermally, intravascularly, or intramuscularly.

17. The method of claim 11, wherein cellular immune reaction against SSX-2 is induced.

18. The method of claim 17, wherein both humoral and cellular immune reactions against SSX-2 are induced.

19. A DNA vaccine comprising a plasmid vector comprising a polynucleotide sequence encoding an SSX-2 polypeptide or an immunogenic fragment thereof operably linked to a transcription regulatory element, wherein upon administration of said vaccine to a mammal a cytotoxic immune reaction against cells expressing SSX-2 is induced.

20. The DNA vaccine of claim 19, wherein the vaccine is suitable for intradermal, intravascular, or intramuscular administration to a human.

21. The DNA vaccine of claim 19, wherein the plasmid vector comprises a backbone of pNGVL3; a polynucleotide sequence encoding an SSX-2 polypeptide or an immunogenic fragment thereof operably inserted therein; and an ISS motif.

22. The DNA vaccine of claim 19, wherein the plasmid vector comprises a polynucleotide sequence encoding an SSX-2 polypeptide or an immunogenic fragment thereof operatively linked to a CMV promoter; a CMV intron A operatively linked to the polynucleotide sequence encoding the SSX-2 polypeptide for enhancing expression of the polynucleotide sequence; and at least one copy of an immunostimulatory fragment comprising 5′-GTCGTT-3′.

23. The DNA vaccine of claim 22, wherein the plasmid vector comprises at least two copies of an immunostimulatory fragment comprising 5′-GTCGTT-3′.

24. The DNA vaccine of claim 19, wherein the plasmid vector does not express in eukaryotic cells any gene other than the polynucleotide sequence encoding the SSX-2 polypeptide or an immunogenic fragment thereof.

25. The DNA vaccine of claim 19, wherein the plasmid vector is pTVG4.

26. A pharmaceutical composition comprising the DNA vaccine of claim 19, and a pharmaceutically acceptable carrier.

27. A pharmaceutical composition comprising the DNA vaccine of claim 19, further comprising a suitable amount of GM-CSF.

28. A pharmaceutical composition comprising the DNA vaccine of claim 22, and a pharmaceutically acceptable carrier.

29. A kit comprising the DNA vaccine of claim 19 and an instruction manual directing administering the vaccine to a mammal having prostate cancer.


SEQUENCE LISTING (8 sequences)

SEQ ID NO: 1 - DNA, 1282 bp, Homo sapiens
  CDS (71)..(637)
ctcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcagagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggacccaaaaggggggaacatgcctggacccacagactgcctgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagattttttcctctgcatttacacacacacgcacacaaaccacaccacacacacacacacacacacacacacacacacacacacaccaagtaccagtataagcatctgccatctgcttttcccattgccatgcgtcctggtcaagctcccctcactctgtttcctggtcagcatgtactcccctcatccgattcccctgtagcagtcactgacagttaataaacctttgcaaacgttcaaaaaaaaaaaaaaaaaaaa

SEQ ID NO: 2 - protein, 188 aa, Homo sapiens
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGPKRGEHAWTHRLPERKQLVIYEEISDPEEDDE

SEQ ID NO: 3 - DNA, 567 bp, Rattus norvegicus
  CDS (1)..(567)
atgaacggagacgacacctttgcaaagagacccagggatgatgctaaagcatcagagaagagaagcaaggcctttgatgatattgccacatacttctctaagaaagagtggaaaaagatgaaatactcggagaaaatcagctatgtgtatatgaagagaaactataaggccatgactaaactaggtttcaaagtcaccctcccacctttcatgtgtaataaacaggccacagacttccaggggaatgattttgataatgaccataaccgcaggattcaggttgaacatcctcagatgactttcggcaggctccacagaatcatcccgaagatcatgcccaagaagccagcagaggacgaaaatgattcgaagggagtgtcagaagcatctggcccacaaaacgatgggaaacaactgcaccccccaggaaaagcaaatatttctgagaagattaataagagatctggacccaaaagggggaaacatgcctggacccacagactgcgtgagagaaagcagctggtgatttatgaagagatcagcgaccctgaggaagatgacgagtaa

SEQ ID NO: 4 - protein, 188 aa, Rattus norvegicus
MNGDDTFAKRPRDDAKASEKRSKAFDDIATYFSKKEWKKMKYSEKISYVYMKRNYKAMTKLGFKVTLPPFMCNKQATDFQGNDFDNDHNRRIQVEHPQMTFGRLHRIIPKIMPKKPAEDENDSKGVSEASGPQNDGKQLHPPGKANISEKINKRSGPKRGKHAWTHRLRERKQLVIYEEISDPEEDDE

SEQ ID NO: 5 - DNA, 928 bp, Mus musculus
  CDS (249)..(746)
gagttaccagaggaattcaaagggtttggctgccctggttgctgagtctctagcaacagctgaatccgggtgcttatttgggtctcttcttggataaacactcctttgtcccttcctgtctgagctcagcatcgccatatagccatttcctaggtccctggaaaccatcagttgggacagggggactattaaatctgtggcaatcctctcaccaagtatatattggacacaccacaaaagatctacacatgatgcagtgcactgtactacaaaatggtgatctaaattccttaaaacgcaggacatttggttttgtgcttgaagaagacaagattgtgtcttttcaaaatcaccctgacatggaaacagtgagttcctgtgaaaagaaccccatggaagtgctttatgaaccaaagaatatctgcaaggccttccaggatatttctacatacttctctgatgaagagtggggaaagctgactcaatggcagaaaagtgcctacgtgtacatgaaaagaaactacatcagaatgactgacctaggggtcaccgtgaaccaaccggttttcatgcgtggtaaggagcaggacaagcaatccctggttgaaggcattgaagttcatgacagtgatgaaactagtggcattcgggtcaatgtttggagccaccggctgcgagagaggaagtatcgagtgatatatgaagagattagtgatcctgaagaggaagaagacgatgattactgaaccaacagtcgaaataagcccccggtggaagaaggatggagtggcgattcaacatcacagctacagctttgaaagactaaatatcagcagtatagaaacatacatttatttatatatttattgatgatatacacttatacctatatttatttatttataaaagtctactggaattctgagtt

SEQ ID NO: 6 - protein, 165 aa, Mus musculus
MMQCTVLQNGDLNSLKRRTFGFVLEEDKIVSFQNHPDMETVSSCEKNPMEVLYEPKNICKAFQDISTYFSDEEWGKLTQWQKSAYVYMKRNYIRMTDLGVTVNQPVFMRGKEQDKQSLVEGIEVHDSDETSGIRVNVWSHRLRERKYRVIYEEISDPEEEEDDDY

SEQ ID NO: 7 - protein, 9 aa, Artificial
  Synthetic peptide
GILGFVFTL

SEQ ID NO: 8 - protein, 9 aa, Artificial
  Synthetic peptide
RLQGISPKI